(19) United States
(12) Patent Application Publication
(10) Pub. No.: US 2023/0080241 A1
(43) Pub. Date: Mar. 16, 2023

(54) METHODS AND COMPOSITIONS FOR ANTIMICROBIAL USE OF SYNTHETIC LYSINE ANALOGS, DERIVATIVES, AND MIMETICS, AND PRODRUGS

(71) Applicant: Tranexamic Technologies, LLC, Dallas, TX (US)

(72) Inventors: W Paul Stewart, Dallas, TX (US); Frank Murdock, Dallas, TX (US)

(21) Appl. No.: 17/799,550

(22) PCT Filed: Jan. 30, 2021
(86) PCT No.: PCT/US2021/015953

§ 371 Date: Aug. 12, 2022

Related U.S. Application Data
(60) Provisional application No. 62/976,723, filed on Feb. 14, 2020.

(51) Int. Cl. A61K 31/454; A61K 45/06; A61K 31/196; A61K 31/198; A61K 9/00

(52) U.S. Cl. CPC A61K 31/454; A61K 45/06; A61K 31/196; A61K 31/198; A61K 9/0014

(57) ABSTRACT

In an embodiment, the present disclosure relates to a method to prevent or inhibit proliferation, growth and formation, or survival of protozoans, bacteria, or fungal cells. In some embodiments, the method includes administering a composition including a synthetic lysine analog, derivative, mimetic, or prodmg. In some embodiments, the synthetic lysine analog, derivative, mimetic, or prodmg interacts with the protozoans, bacteria, or fungal cells to prevent or inhibit proliferation, growth and formation, or survival of the protozoans, bacteria, or fungal cells. In an additional embodiment, the present disclosure relates to a composition to prevent or inhibit proliferation, growth and formation, or survival of protozoans, bacteria, or fungal cells. In some embodiments, the composition includes a synthetic lysine analog, derivative, mimetic, or prodrug.

### CROSS-REFERENCE TO RELATED APPLICATIONS

This patent application claims priority from, and incorporates by reference the entire disclosure of, U.S. Provisional Application No. 62/976,723 filed on Feb. 14, 2020.

### TECHNICAL FIELD

The present disclosure relates generally to antimicrobial agents and more particularly, but not by way of limitation, to compositions and methods for antimicrobial use of synthetic lysine analogs, derivatives, mimetics, and prodrugs.

### BACKGROUND

This section provides background information to facilitate a better understanding of the various aspects of the disclosure. It should be understood that the statements in this section of this document are to be read in this light, and not as admissions of prior art.

Plasmodium falciparum is a unicellular protozoan parasite, and the deadliest species of Plasmodium that causes malaria in humans. The parasite is transmitted through the bite of Anopheles mosquitos and causes the disease's most dangerous form called falciparum malaria which is responsible for around half of all malaria cases. P. falciparum is typically regarded as one of the deadliest parasites to humans, and has caused more than 400,000 deaths per year. The World Health Organization World Malaria Report disclosed over 200 million cases of malaria worldwide in a previous year, resulting in an estimated 405,000 deaths that year. Almost every malarial death in humans is caused by P. falciparum, and children under five years of age are most affected, accounting for about 61% of the total deaths.

Additionally, Cryptosporidium is a microscopic parasite that causes the diarrheal disease cryptosporidiosis. Both the parasite and the disease are commonly referred to as crypto. There are many species of Cryptosporidium that infect humans. Cryptosporidiosis is a parasitic disease caused by Cryptosporidium, a genus of protozoan parasites in the phylum Apicomplexa, and is estimated to cause more than 200,000 deaths a year. It affects the distal small intestine and can affect the respiratory tract in both immunocompetent (i.e., individuals with a normal functioning immune system) and immunocompromised (i.e., individuals with an impaired immune system) individuals, resulting in watery diarrhea, with or without an accompanied and unexplained cough. In immunosuppressed individuals, the symptoms are particularly severe and can often be fatal. The parasite is protected by an outer shell that allows it to survive outside the body for prolonged periods of time and makes it very tolerant to chlorine disinfection. While this parasite can be spread in several different ways, drinking water and recreational water are the most common ways the parasite spreads. This makes Cryptosporidium a leading cause of waterborne disease among humans.

However, malaria and cryptosporidiosis are only two infectious disease among an ever-growing list of infectious diseases that interfere with human activity. While protozoans account for a large amount of infectious diseases, bacteria and fungi also cause many diseases among the human population. For example, bacterial meningitis, otitis media, pneumonia, and tuberculosis make up a large number of bacterial infections in humans. Additionally, tinea pedis (athlete's foot), tinea corporis (ring worm), and cutaneous candidiasis are only few of the many fungal infections that infect humans globally.

Some protozoan, bacterial, or fungal infections are mild and can even be unnoticeable. However, others, such as malaria or cryptosporidiosis, can be severe and life-threatening. Furthermore, some of the protozoans, bacteria, or fungi are becoming resistant to currently available treatments. The spread of drug resistance is a growing concern for malaria treatment, and there is currently no effective treatment for malnourished or immunocompromised children infected with Cryptosporidium.

Infection of these diseases can be transmitted in a variety of ways, and can include, without limitation, skin contact, contact with feces, bodily fluid exchange, contact with airborne particles, or combinations of the same and like. Additionally, merely touching an object that an infected person has previously been in contact with can cause infection in an individual. As such, new prevention and treatment compositions and methods with novel mechanisms of action are needed for malaria, cryptosporidiosis, and other protozoans, in addition to bacterial or fungal infections. In view of the aforementioned, antimicrobials are needed to inhibit and prevent protozoan, bacterial, or fungal infections. Various embodiments of the present disclosure seek to address this need.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure relates to a method to prevent or inhibit proliferation, growth and formation, or survival of protozoans, bacteria, or fungal cells. Generally, the method includes administering a composition including a synthetic lysine analog, derivative, mimetic, or prodrug. In some embodiments, the synthetic lysine analog, derivative, mimetic, or prodrug interacts with the protozoans, bacteria, or fungal cells to prevent or inhibit proliferation, growth and formation, or survival of the protozoans, bacteria, or fungal cells.

In an additional embodiment, the present disclosure relates to a composition to prevent or inhibit proliferation, growth and formation, or survival of protozoans, bacteria, or fungal cells. In some embodiments, the composition includes a synthetic lysine analog, derivative, mimetic, or prodrug. In some embodiments, the synthetic lysine analog, derivative, mimetic, or prodrug interacts with the protozoans, bacteria, or fungal cells to prevent or inhibit proliferation, growth and formation, or survival of the protozoans, bacteria, or fungal cells.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates in vitro asexual blood stage growth of Plasmodium falciparum at day 1 after 1 dose of tranexamic acid (TXA).

FIG. 2 illustrates in vitro asexual blood stage growth of P. falciparum at day 3 after 3 doses of tranexamic acid (TXA).

FIG. 3 illustrates P. falciparum in vitro IC₅₀ of tranexamic acid after three daily doses.

FIG. 4 illustrates in vivo asexual blood stage growth of Plasmodium berghei after the injection of a phosphate-buffered saline (PBS) carrier.

FIG. 5 illustrates in vivo asexual blood stage growth of P. berghei after the injection of 10 mg of tranexamic acid.

FIG. 6 illustrates in vivo asexual blood stage growth of P. berghei after the injection of 20 mg of tranexamic acid.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

For proliferation and survival, protozoans, bacteria, and fungi, require protein-protein, protein-DNA, and protein-RNA interactions involving the three positively charged amino acids lysine, arginine, and histidine. These three positively charged amino acids (lysine, arginine, and histidine) provide the positive charge in the electrostatic bond between positively and negatively charged residues required in multiple protein-protein, protein-DNA, and protein-RNA connections involved in various biological activities such as, but not limited to, the proliferation and survival of protozoans, bacteria, and fungi. These three amino acids have similar chemical structures. As such, adding supplemental amounts of one or more can block the activity of one or more of the amino acids that are not supplemented. Similarly, providing a sufficient amount of an appropriate synthetic analog, derivative, mimetic, or prodrug of lysine, arginine, or histidine can block the activity of one or more of the three amino acids. In brief, synthetic lysine analogs, derivatives, mimetics, or prodrugs (herein referred to as “lysine analogs”) can disrupt the protein-protein, protein-DNA, and protein-RNA interactions by antagonizing lysine, arginine, and histidine residues on the proteins.

As protein-protein, protein-DNA, and protein-RNA interactions are of importance for cells to grow, multiply, and survive, lysine analogs are of great interest for antimicrobial benefits. For example, tranexamic acid, an example of a lysine analog, inhibits protozoans, bacteria, and fungi growth by occupying binding sites of lysine residues required for cell growth and replication, similar to how lysine analogs prevent plasminogen from converting to plasmin.

Studies have shown a series of antimalarial bicyclic azetidines, identified by phenotypic screening, that were found to inhibit cytosolic Plasmodium phenylalanyl-tRNA synthetase. The compounds in this study showed activity across multiple life stages of the parasite and in vivo efficacy in malaria models. Malaria parasite genomes encode two different lysyl-tRNA synthetases (KRSs) that play a role in translation in either the cytoplasm (PfKRS1) or in the apicoplast (PfKRS2), while Cryptosporidium parasites and humans encode one copy. Human KRS (HsKRS) is found in both the cytosol and mitochondrion and has additional roles within human cells. Studies of the inhibition of PfKRS1 by various compounds have been performed using pyrophosphate generation platforms. To study the mechanism of inhibition by these compounds, single-inhibition measurements were performed at a fixed saturating concentration of one substrate and fixed variable concentrations of a second substrate. Under these conditions, results indicated that the studied compounds compete with adenosine triphosphate (ATP) for the same binding site and only binds in the presence of L-lysine. The results indicate that, in the presence of high concentrations of ATP, the binding affinity of the compounds are reduced, whereas in the presence of high concentrations of L-lysine, the binding affinity is increased. With respect to PfKRS1, simulations performed in the absence of L-lysine showed a notable destabilization of the compounds, suggesting a role of L-lysine in the binding of PfKRS1 inhibitors. Furthermore, the Cryptosporidium lysyl-tRNA synthetase (CpKRS) system exhibited behavior similar to PfKRS1, demonstrating an affinity toward CpKRS.

This study represents strong validation of lysyl-tRNA synthetase as a target in both malaria and cryptosporidiosis due to the use of lysine in the cell growth process. Accordingly, antimicrobial agents that inhibit or prevent lysine in the growth process of cells will prevent the proliferation and facilitate in the ultimate death of the cells needing lysine for growth. As such, the lysine analogs presented herein can prevent the growth and proliferation of malaria and cryptosporidiosis by inhibiting lysine binding required in cell growth. Generally, these principals further apply to other types of protozoans, bacteria, and fungi with similar binding activity.

Additionally, studies have shown that the meso-diaminopimelate (m-DAP)/lysine biosynthetic pathway offers several potential antibacterial enzyme targets. Lysine is one of the products of this pathway, and is required in protein synthesis. Lysine is also used in the peptidoglycan layer of Gram-positive bacterial cell walls. A second product of this pathway, m-DAP, is a component of the peptidoglycan cell wall for Gram-negative bacteria, providing a link between poly-saccharide strands. Most bacteria synthesize lysine and m-DAP from aspartic acid through three related pathways that diverge after the production of L-tetrahydrodipicolinate. Findings have shown that the presence of multiple biosynthetic pathways in bacteria for the synthesis of m-DAP/lysine highlights the importance of m-DAP/lysine for bacterial cell survival and growth. Research has shown that the succinylase pathway is the primary biosynthetic pathway for m-DAP/lysine and is used by Gram-negative and Gram-positive bacteria. The dehydrogenase pathway forms m-DAP directly from L-tetrahydrodipicolinate, but this is a high-energy transformation and is limited to only a few bacterial species belonging to the Bacillus class. The acetylase pathway is also a minor biosynthetic pathway for m-DAP production, and is also limited to only a few Bacillus species. However, one of the enzymes in the succinylase pathway, the dapE-encoded N-succinyl-L,L-diaminopimelic acid desuccinylase (DapE), is a Zn(II)-containing metallohydrolase, and it has been shown that deletion of the gene encoding DapE is lethal to certain bacteria. Even in the presence of lysine-supplemented media, certain bacteria is unable to grow, suggesting that lysine cannot be synthesized by other pathways or be imported. Therefore, DapE enzymes appear to play a role in cell growth and proliferation, and are part of a biosynthetic pathway that is the only source of lysine in most bacteria. This research suggests that DapE enzymes appear to be potential targets for inhibitors that may possess antimicrobial activity, especially in view that there are no similar biosynthetic pathways in humans.

In view of this, removing lysine from biosynthetic pathways via lysine analogs offer a high potential for antimicrobial properties. The above illustrates the need for lysine in bacterial proliferation, thus providing a sufficient amount of an appropriate lysine analog would lead to blocking the activity of lysine. As such, synthetic lysine analogs would inhibit lysine in the biosynthetic pathway to thereby prevent the growth and proliferation of bacteria. Moreover, these principals apply generally to protozoans and fungi with similar biosynthetic pathways.

In view of the aforementioned, lysine analogs can be utilized as antimicrobial agents due to their ability to disrupt the protein-protein, protein-DNA, and protein-RNA interactions by antagonizing lysine, arginine, and histidine residues of proteins and removing lysine from biosynthetic pathways. As lysine analogs, such as, for example, tranexamic acid, can occupy the binding sites of lysine in the growth process of protozoans, bacteria, and fungi, or remove lysine from the biosynthetic pathways, lysine analogs would be an advantageous avenue for therapy against various diseases, such as, but not limited to, malaria and cryptosporidiosis. Owing to the fact that lysine analogs, such as tranexamic acid, have very low toxicity, utilizing lysine analogs can be an effective treatment for protozoan, bacterial, and fungal infections even for infections that may require a high dosage of tranexamic acid.

Furthermore, lysine analogs can be used prophylactically as routine preventative care for individuals who are frequently near or around other individuals with the potential of having a protozoan, bacterial, or fungal infection. These individuals can include, among others, nurses, doctors, teachers, frequent travelers, or other people generally in recurrent contact with a large number of other people. Additionally, lysine analogs can be utilized prophylactically in immunocompromised individuals, or other high-risk individuals, to prevent infection due to protozoans, bacteria, or fungi. This would be advantageous for individuals at greater risk of severe consequences from infection, such as the elderly or infants, in addition to individuals at heightened risk of exposure to infection.

As lysine analogs have many advantageous properties in preventing or inhibiting proliferation, growth and formation, or survival of protozoans, bacteria, or fungal cells, lysine analogs, such as tranexamic acid, can be used in combination other antimicrobial therapeutic agents. In some instances, it is advantageous to combine lysine analogs with various therapeutic agents with differing methods of action. In this manner, protozoans, bacteria, or fungal cells can be combatted via multiple modes and mechanisms. Additionally, other therapeutic agents can have varying activity in which their effects are complimentary to lysine analogs. Furthermore, an additional therapeutic agent, when combined with lysine analogs, can provide for a lower dose of either the lysine analog or the additional therapeutic agent. In some instances, a combined effect can be greater than that predicted by individual potencies of each individual constituent, for example, either by requiring lower concentrations or by reacting more positively at similar concentrations. These interactions allow, for example, the use of lower concentrations of each constituent, a situation that can reduce adverse reactions of each individual constituent. Additionally, antimalarial drugs, antibiotics, and antifungal drugs suffer from a reduction in effectiveness of the antimalarial drugs, antibiotics, and antifungal drugs (i.e., drug resistance). As such, by using a combination of therapeutic agents, these reduced-efficacy drugs can be made more effective with the addition of lysine analogs, such as, but not limited to, tranexamic acid. In some instances, the combination of therapeutic agents, for example, antimalarial drugs, antibiotics, and antifungal drugs with lysine analogs, such as tranexamic acid, can provide for a synergistic effect, thus making the treatment of infection more effective, while also avoiding drug resistance.

Further, secondary effects of one of the constituents can enhance the primary effect of another constituent, or provide other benefits such as, but not limited to, enhanced immune response and a greater inhibition of proliferation, growth and formation, or survival of protozoans, bacteria, or fungal cells. For example, lysine analogs can be combined with antimalarial drugs, antibiotics, and antifungal drugs to provide antifibrinolytic effects in individuals who are suffering from protozoan, bacterial, or fungal infections where bleeding occurs. Moreover, lysine analogs can be combined with antimalarial drugs, antibiotics, and antifungals to provide anti-inflammatory and immune enhancement effects provided by the lysine analogs. These secondary benefits can be highly advantageous in the treatment of protozoan, bacterial, or fungal infections.

Reference will now be made to more specific embodiments of the present disclosure and data that provides support for such embodiments. However, it should be noted that the disclosure below is for illustrative purposes only and is not intended to limit the scope of the claimed subject matter in any way.

Presented herein is experiment data representing tranexamic acid as an antimalarial druggable scaffold. The results shown below show the efficacy of tranexamic acid against both Plasmodium falciparum and P. berghei across the parasite lifecycle. The data herein illustrates the ability of tranexamic acid to act as an antimicrobial agent, and more particularly, in some embodiments, as an antimalarial drug. Experiments were conducted to test, in vitro, the ability of tranexamic acid to impair the asexual intraerythrocytic development of P. falciparum. All of the symptoms associated with malaria infection are caused by the asexual blood stages, which makes them a logical therapeutic target.

Up to six different concentrations of tranexamic acid plus a negative control (carrier with no drug) were tested in a highly synchronous culture. A two-fold serial dilution starting at 200 mM was prepared, replenishing the drug daily for four consecutive days (i.e. two complete intraerythrocytic cycles).

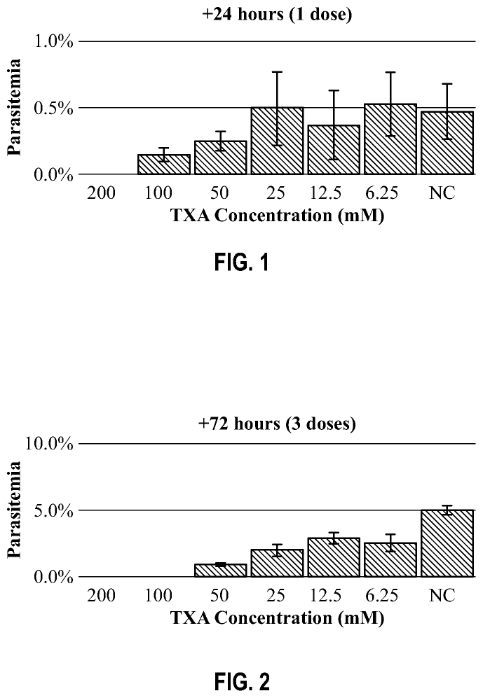

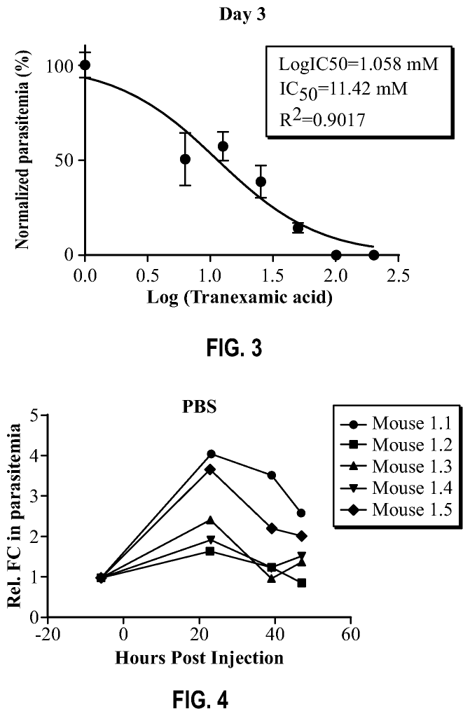

FIG. 1 illustrates in vitro asexual blood stage growth at day 1 after 1 dose of tranexamic acid (TXA). Values are the mean of three technical replicates, and error bars represent the standard deviation. FIG. 2 illustrates in vitro asexual blood stage growth at day 3 after 3 doses of tranexamic acid (TXA). Values are the mean of three technical replicates, and error bars represent the standard deviation. FIG. 3 illustrates in vitro IC₅₀ of tranexamic acid after three daily doses. Values are the mean of three technical replicates, relative to the minimal effect (i. e. the parasitemia from the samples with no drug). Error bars represent the standard deviation. Morphological defective parasites could be observed at day 4 in the two lowest tested concentrations (i.e. 12.5 and 6.25 mM). Healthy segmented schizonts were selected from the sample mock-treated in the absence of tranexamic acid (0 mM).

Based on the data presented herein, 200 mM tranexamic acid completely cleared the infection after 24 hours (i.e. with a single dose, as shown in FIG. 1), while the 100 mM cleared the infection after 48 hours (two doses). Additionally, a growth delay was observed in some of the tested concentrations. Rings and early trophozoites were observed on day 3 (i.e. 72 hours after the first dose of the drug), while not a single ring stage could be observed in the negative control with no drug (the culture was synchronized with sorbitol for two consecutive cycles prior to the experiment), as demonstrated in FIG. 2. Furthermore, the IC₅₀ at day 3 (i.e. after 3 doses) was 11.42 nM, as illustrated in FIG. 3. Moreover, morphological defects could be observed at day 4 in the samples treated with 12.5 and 6.25 mM tranexamic acid (some of the parasites were arrested at a “schizont-like” morphology).

In addition to in vitro experiments to test the ability of tranexamic acid to impair the asexual intraerythrocytic development of P. falciparum, experiments were conducted to test, in vivo, the ability of tranexamic acid to cure asynchronized blood stage infections of Plasmodium berghei ANKA mCherry in mice. P. berghei is a species in the genus Plasmodium subgenus Vinckeia and is a protozoan parasite that causes malaria in rodents. Due to its ability to infect rodents and relative ease of genetic engineering, P. berghei is a popular model organism for the study of human malaria, and was thus used in the present disclosure.

Recipient mice (n=5/group; 3 groups) received blood from a donor mouse containing asynchronous asexual blood stages of P. berguei. Parasitemia was monitored and when it reached at least 5% mice were treated intravenously (i.v.) with 20, 10, or 0 mg of tranexamic acid in a phosphate-buffered saline (PBS) carrier. Blood smears from tail snips were prepared every 24 hours, or until the humane endpoint criteria was reached, to monitor parasitemia.

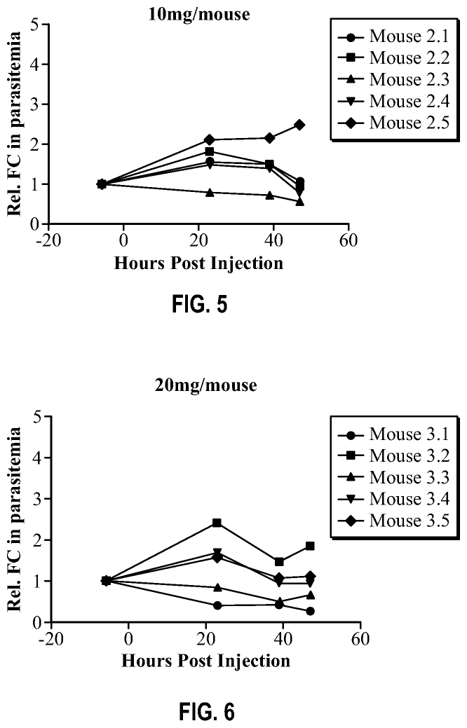

FIG. 4 illustrates in vivo asexual blood stage growth of P. berghei after the injection of the PBS carrier. The Y-axis represents the parasitemia of each mouse relative to its corresponding initial parasitemia (fold-change) before the injection of the PBS carrier (control/sham treatment), and the X-axis represents the hours after the injection (i.v.) of tranexamic acid. FIG. 5 illustrates in vivo asexual blood stage growth of P. berghei after the injection of 10 mg of tranexamic acid. The Y-axis represents the parasitemia of each mouse relative to its corresponding initial parasitemia (fold-change) before the injection of 10 mg of tranexamic acid (i.v.), and the X-axis represents the hours after the injection. FIG. 6 illustrates in vivo asexual blood stage growth of P. berghei after the injection of 20 mg of tranexamic acid. The Y-axis represents the parasitemia of each mouse relative to its corresponding initial parasitemia (fold-change) before the injection of 20 mg of tranexamic acid (i.v.), and the X-axis represents the hours after the injection.

This data demonstrates P. berghei in vivo parasite growth seems to be either maintained at the initial parasitemia (i.e. parasitemia, which is expected to increase, is “checked”) or reduced in a few of the mice treated with tranexamic acid (either 20 or 10 mg) if compared with mice injected with the PBS carrier (this is more evident at 20 hours post injection). Experiments presented in the present disclosure utilized outbred CD1 mice, and despite using a common stock bolus of parasites (received from the donor mouse) to initiate infection, each mouse is expected to experience a differential malaria pathogenesis due to natural physiological differences, such as, for example, reticulocytemia. Reticulocytes are the preferred cell for infection by P. berghei merozoites. Based on the estimated volemia of the mice, 10 and 20 mg tranexamic acid injections are equivalent to approximately 37 and 75 mM tranexamic acid concentrations in vitro, respectively. Both concentrations are above the in vitro IC₅₀ calculated in previous experiments when the drug was replenished on a daily basis (i.e. 11.42 mM).

Based on the data presented herein, lysine analogs can be administered in a subject infected with a protozoan, bacterial, or fungal infection via various modes of delivery and at varying dosages. For instance, delivery modes can include, but are not limited to, intravenous delivery, oral delivery, topical delivery, or combinations thereof. As an example, the lysine analog can be administered intravenously to the subject in an amount that produces a concentration in the subject of about 10 to 100 mg/kg by weight of the subject every 8 hours, and the administration can be over a period of time including, but not limited to, 5 to 7 days or until the protozoan, bacterial, or fungal infection is resolved. In various instances, the lysine analog can be administered intravenously to the subject in an amount that produces a concentration in the subject of about 37 to 75 mM every 8 hours, and the administration can be over a period of time including, but not limited to, 5 to 7 days or until the protozoan, bacterial, or fungal infection is resolved. In some embodiments, the intravenous administration can occur as repeat boluses every 8 hours. In some embodiments, the intravenous administration can occur with an initial bolus followed by a continuous infusion of approximately one-tenth of the initial bolus per hour of the lysine analog for a requisite amount of time, for example, until the protozoan, bacterial, or fungal infection has resolved.

Additionally, the lysine analog can be administered orally to the subject, in the form of a pill, tablet, capsule, or an oral solution or syrup such that the oral administration produces a concentration in the subject of about 20 to 200 mg/kg by weight of the subject every 8 hours, and the administration can be over a period of time including, but not limited to, 5 to 7 days or until the protozoan, bacterial, or fungal infection is resolved. Moreover, the lysine analog can have higher or lower concentrations, depending on what type of protozoan, bacterial, or fungal infection the subject has.

It is readily envisioned that higher and lower doses may be utilized, administration times and duration can be shortened or extended, and varying delivery modes can ensue. For example, a lysine analog can be delivered topically to the skin of a subject. This can be advantageous when the protozoan, bacterial, or fungal infection is on the skin, for example, if the subject is suffering from leishmaniasis, a disease caused by a protozoan from the genus Leishmania. In this example, the lysine analog can be in the form of a gel or cream readily adaptable to be applied to the skin, and have a concentration in a range of approximately 1 to 30% by weight of the lysine analog. In some embodiments, the concentration of the gel or cream can be approximately 20% by weight of the lysine analog. As some lysine analogs remain in skin tissue, providing therapeutic benefits for about 8 hours, topical delivery can occur every 8 hours, until the infection is resolved. Accordingly, various doses and administration for different types of protozoans, bacterial, and fungal infections are readily envisioned.

In view of the aforementioned, synthetic lysine analogs, derivatives, mimetics, or prodrugs (“lysine analogs”) exhibit advantageous properties as antimicrobials, as the lysine analogs inhibit the growth of protozoans, bacteria, and fungi. As disclosed herein, a specific lysine analog, tranexamic acid, has been show to clear malaria infection, thus demonstrating its ability to act as an antimicrobial agent. Lysine analogs, and specifically, tranexamic acid, have been demonstrated to disrupt protein-protein, protein-DNA, and protein-RNA interactions by antagonizing lysine, arginine, and histidine residues on the proteins. In addition, lysine analogs can further inhibit lysine in the biosynthetic pathway to thereby prevent the growth and proliferation of cells, and as such, can be utilized in the treatment and prevention of protozoan, bacterial, and fungal infections. As disclosed herein, a specific lysine analog, tranexamic acid, has been shown to disrupt the growth, proliferation, and survival of malaria, demonstrating the exceptional behavior of tranexamic acid as an antimicrobial agent. In addition to P. falciparum and P. berghei protozoans, lysine analogs, such as, but not limited to, tranexamic acid are envisioned to exhibit antimicrobial effects on bacteria, such as, but not limited to, Methicillin-resistant Staphylococcus aureus, yeast-type fungi, such as, but not limited to, Candida auris and Saccharomyces boulardi, and mold-type fungi such as, but not limited to, Trichophyton interdigitale. Various other types of protozoans, bacteria, and fungi that have similar cell growth and proliferation mechanisms are readily envisioned.

As such, in an embodiment, the present disclosure relates to a method to prevent or inhibit proliferation, growth and formation, or survival of protozoans, bacteria, or fungal cells. In some embodiments, the method includes administering a composition including a synthetic lysine analog, derivative, mimetic, or prodrug. In some embodiments, the synthetic lysine analog, derivative, mimetic, or prodrug interacts with the protozoans, bacteria, or fungal cells to prevent or inhibit proliferation, growth and formation, or survival of the protozoans, bacteria, or fungal cells.

In some embodiments, the method includes disrupting, by the composition, at least one of protein-protein, protein-DNA, or protein-RNA interaction by antagonizing at least one of lysine, arginine, or histidine residues on a protein. In some embodiments, the method includes inhibiting, by the composition, proliferation, growth and formation, or survival of the protozoans, bacteria, or fungal cells by occupying binding sites of lysine residues required for proliferation, growth and formation, or survival of the protozoans, bacteria, or fungal cells. In some embodiments, the method includes removing, by the composition, lysine from a biosynthetic pathway required by the protozoans, bacteria, or fungal cells to thereby inhibit proliferation, growth and formation, or survival of the protozoans, bacteria, or fungal cells.

In some embodiments, the synthetic lysine analog, derivative, mimetic, or prodrug can include, without limitation, tranexamic acid, epsilon-aminocaproic acid (EACA), or AZD 6564. In some embodiments, the protozoans, bacteria, or fungal cells can include, without limitation, Plasmodium falciparum, Plasmodium berghei, Methicillin-resistant Staphylococcus aureus, Candida auris, Saccharomyces boulardi, Trichophyton interdigitale, Leishmania, or combinations thereof. In some embodiments, the protozoans, bacteria, or fungal cells can include, without limitation, P. falciparum or P. berghei.

In some embodiments, the composition is in a solution. In some embodiments, the solution contains an amount that provides about 37 mM concentration of the synthetic lysine analog, derivative, mimetic, or prodrug in a subject. In some embodiments, the solution contains an amount that provides about 75 mM concentration of the synthetic lysine analog, derivative, or mimetic, or prodrug in a subject.

In some embodiments, the solution is administered intravenously. In some embodiments, the solution contains an amount that provides up to about 100 mg/kg concentration by weight of a subject of the lysine analog, derivative, mimetic, or prodrug in the subject. In some embodiments, the solution contains an amount that provides about 10 mg/kg concentration by weight of a subject of the lysine analog, derivative, mimetic, or prodrug in the subject. In some embodiments, the solution is administered every 8 hours. In some embodiments, the solution is administered as an initial bolus followed by continuous infusion for a requisite period of time. In some embodiments, the continuous infusion is approximately one-tenth of the initial bolus per hour. In some embodiments, the solution is administered for approximately 5 to 7 days. In some embodiments, the solution is administered until an infection caused by the protozoans, bacteria, or fungal cells has been resolved.

In some embodiments, the solution is applied as part of a vehicle which adapts to human skin. In some embodiments, the solution is in a gel or cream formation. In some embodiments, the solution has a concentration of about 1 to 30% by weight of the synthetic lysine analog, derivative, mimetic, or prodrug. In some embodiments, the solution has a concentration of about 20% by weight of the synthetic lysine analog, derivative, mimetic, or prodrug. In some embodiments, the solution is administered topically every 8 hours. In some embodiments, the solution is administered topically until an infection caused by the protozoans, bacteria, or fungal cells has been resolved. In some embodiments, the solution is administered via a vehicle that allows the synthetic lysine analog, derivative, mimetic, or prodrug to be delivered in a time-released fashion.

In some embodiments, the composition is delivered orally. In some embodiments, the composition is in a form of at least one of a pill, a tablet, a capsule, or an oral solution or syrup. In some embodiments, the composition includes the synthetic lysine analog, derivative, mimetic, or prodrug in an amount that provides up to about 200 mg/kg concentration by weight of a subject of the lysine analog, derivative, mimetic, or prodrug in the subject. In some embodiments, the composition includes the synthetic lysine analog, derivative, mimetic, or prodrug in an amount that provides about 20 mg/kg concentration by weight of a subject of the lysine analog, derivative, mimetic, or prodrug in the subject. In some embodiments, the composition is administered every 8 hours. In some embodiments, the composition is administered for approximately 5 to 7 days. In some embodiments, the composition is administered until an infection caused by the protozoans, bacteria, or fungal cells has been resolved. In some embodiments, the composition is delivered in a time-released fashion.

In some embodiments, the administering of the composition occurs at least once a day. In some embodiments, the composition is administered by at least one of a mechanical device, a permanent or resorbable material, or a vehicle whereby the synthetic lysine analog, derivative, mimetic, or prodrug is delivered in a time-released fashion. In some embodiments, the composition is administered via a transdermal patch. In some embodiments, the composition is administered via an injected or implanted liposomal delivery depot. In some embodiments, the composition includes ingredients that provide for rapid systemic penetration or extended release. In some embodiments, the administering is performed systemically.

In some embodiments, the composition includes an additional therapeutic agent. In some embodiments, the additional therapeutic agent has a mechanism of action complimentary to the synthetic lysine analog, derivative, mimetic, or prodrug. In some embodiments, the synthetic lysine analog, derivative, mimetic, or prodrug is a lysine prodrug that causes production of lysine, a lysine analog, a lysine derivative, or a lysine mimetic in a subject. In some embodiments, the synthetic lysine analog, derivative, mimetic, or prodrug is a lysine prodrug that can include, without limitation, a lysine analog prodrug, a lysine derivative prodrug, and a lysine mimetic prodrug.

In an additional embodiment, the present disclosure relates to a composition to prevent or inhibit proliferation, growth and formation, or survival of protozoans, bacteria, or fungal cells. In some embodiments, the composition includes a synthetic lysine analog, derivative, mimetic, or prodrug. In some embodiments, the synthetic lysine analog, derivative, mimetic, or prodrug interacts with the protozoans, bacteria, or fungal cells to prevent or inhibit proliferation, growth and formation, or survival of the protozoans, bacteria, or fungal cells.

In some embodiments, the synthetic lysine analog, derivative, mimetic, or prodrug disrupts at least one of protein-protein, protein-DNA, or protein-RNA interaction by antagonizing at least one of lysine, arginine, or histidine residues on a protein. In some embodiments, the synthetic lysine analog, derivative, mimetic, or prodrug inhibits proliferation, growth and formation, or survival of the protozoans, bacteria, or fungal cells by occupying binding sites of lysine residues required for proliferation, growth and formation, or survival of the protozoans, bacteria, or fungal cells. In some embodiments, the synthetic lysine analog, derivative, mimetic, or prodrug removes lysine from a biosynthetic pathway required by the protozoans, bacteria, or fungal cells to thereby inhibit proliferation, growth and formation, or survival of the protozoans, bacteria, or fungal cells.

In some embodiments, the synthetic lysine analog, derivative, or mimetic can include, without limitation, tranexamic acid, epsilon-aminocaproic acid (EACA), or AZD 6564. In some embodiments, the protozoans, bacteria, or fungal cells can include, without limitation, Plasmodium falciparum, Plasmodium berghei, Methicillin-resistant Staphylococcus aureus, Candida auris, Saccharomyces boulardi, Trichophyton interdigitale, Leishmania, or combinations thereof. In some embodiments, the protozoans, bacteria, or fungal cells can include, without limitation, P. falciparum or P. berghei.

In some embodiments, the synthetic lysine analog, derivative, mimetic, or prodrug is in a solution. In some embodiments, the solution contains an amount that provides about 37 mM concentration of the synthetic lysine analog, derivative, mimetic, or prodrug in a subject. In some embodiments, the solution contains an amount that provides about 75 mM concentration of the synthetic lysine analog, derivative, mimetic, or prodrug in a subject.

In some embodiments, the solution is administered intravenously. In some embodiments, the solution contains an amount that provides up to about 100 mg/kg concentration by weight of a subject of the lysine analog, derivative, mimetic, or prodrug in the subject. In some embodiments, the solution contains an amount that provides about 10 mg/kg concentration by weight of a subject of the lysine analog, derivative, mimetic, or prodrug in the subject. In some embodiments, the solution is administered every 8 hours. In some embodiments, the solution is administered as an initial bolus followed by continuous infusion for a requisite period of time. In some embodiments, the continuous infusion is approximately one-tenth of the initial bolus per hour. In some embodiments, the solution is administered for approximately 5 to 7 days. In some embodiments, the solution is administered until an infection caused by the protozoans, bacteria, or fungal cells has been resolved.

In some embodiments, the solution is applied as part of a vehicle which adapts to human skin. In some embodiments, the solution is in a gel or cream formation. In some embodiments, the solution has a concentration of about 1 to 30% by weight of the synthetic lysine analog, derivative, mimetic, or prodrug. In some embodiments, the solution has a concentration of about 20% by weight of the synthetic lysine analog, derivative, mimetic, or prodrug. In some embodiments, the solution is administered topically every 8 hours. In some embodiments, the solution is administered topically until an infection caused by the protozoans, bacteria, or fungal cells has been resolved. In some embodiments, the solution is administered via a vehicle that allows the synthetic lysine analog, derivative, mimetic, or prodrug to be delivered in a time-released fashion.

In some embodiments, the synthetic lysine analog, derivative, mimetic, or prodrug is delivered orally. In some embodiments, the synthetic lysine analog, derivative, mimetic, or prodrug is in a form of at least one of a pill, a tablet, a capsule, or an oral solution or syrup. In some embodiments, the synthetic lysine analog, derivative, mimetic, or prodrug is in an amount that provides up to about 200 mg/kg concentration by weight of a subject of the lysine analog, derivative, mimetic, or prodrug in the subject. In some embodiments, the synthetic lysine analog, derivative, mimetic, or prodrug is in an amount that provides about 20 mg/kg concentration by weight of a subject of the lysine analog, derivative, mimetic, or prodrug in the subject. In some embodiments, the synthetic lysine analog, derivative, mimetic, or prodrug is administered every 8 hours. In some embodiments, the synthetic lysine analog, derivative, mimetic, or prodrug is administered for approximately 5 to 7 days. In some embodiments, the synthetic lysine analog, derivative, mimetic, or prodrug is administered until an infection caused by the protozoans, bacteria, or fungal cells has been resolved. In some embodiments, the synthetic lysine analog, derivative, mimetic, or prodrug is delivered in a time-released fashion.

In some embodiments, administration of the synthetic lysine analog, derivative, mimetic, or prodrug occurs at least once a day. In some embodiments, the synthetic lysine analog, derivative, mimetic, or prodrug is administered by at least one of a mechanical device, a permanent or resorbable material, or a vehicle whereby the synthetic lysine analog, derivative, mimetic, or prodrug is delivered in a time-released fashion. In some embodiments, the synthetic lysine analog, derivative, mimetic, or prodrug is administered via a transdermal patch. In some embodiments, the synthetic lysine analog, derivative, mimetic, or prodrug is administered via an injected or implanted liposomal delivery depot. In some embodiments, the composition further includes ingredients that provide for rapid systemic penetration or extended release. In some embodiments, administration of the synthetic lysine analog, derivative, mimetic, or prodrug is performed systemically.

In some embodiments, the composition further includes an additional therapeutic agent. In some embodiments, the additional therapeutic agent has a mechanism of action complimentary to the synthetic lysine analog, derivative, mimetic, or prodrug. In some embodiments, the synthetic lysine analog, derivative, mimetic, or prodrug is a lysine prodrug that causes production of lysine, a lysine analog, a lysine derivative, or a lysine mimetic in a subject. In some embodiments, the synthetic lysine analog, derivative, mimetic, or prodrug is a lysine prodrug that can include, without limitation, a lysine analog prodrug, a lysine derivative prodrug, and a lysine mimetic prodrug.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A method to prevent or inhibit proliferation, growth and formation, or survival of protozoans, bacteria, or fungal cells, the method comprising:
administering a composition comprising a synthetic lysine analog, derivative, mimetic, or prodrug, wherein the synthetic lysine analog, derivative, mimetic, or prodrug interacts with the protozoans, bacteria, or fungal cells to prevent or inhibit proliferation, growth and formation, or survival of the protozoans, bacteria, or fungal cells, wherein the administration disrupts at least one of protein-protein, protein-DNA, or protein-RNA interaction by antagonizing at least one of lysine, arginine, or histidine residues on a protein, inhibits proliferation, growth and formation, or survival of the protozoans, bacteria, or fungal cells by occupying binding sites of lysine residues required for proliferation, growth and formation, or survival of the protozoans, bacteria, or fungal cells or deprives lysine or arginine from a biosynthetic pathway required by the protozoans, bacteria, or fungal cells to thereby inhibit proliferation, growth and formation, or survival of the protozoans, bacteria, or fungal cells

2-4. (canceled)

5. The method of claim 1, wherein the synthetic lysine analog, derivative, or mimetic is selected from the group consisting of tranexamic acid, epsilon-aminocaproic acid (EACA), AZD 6564, a lysine prodrug selected from the group consisting of a lysine analog prodrug, a lysine derivative prodrug, and a lysine mimetic prodrug.

6. The method of claim 1, wherein the protozoans, bacteria, or fungal cells are selected from the group consisting of Plasmodium falciparum, Plasmodium berghei, Methicillin-resistant Staphylococcus aureus, Candida auris, Saccharomyces boulardi, Trichophyton interdigitale, Leishmania, and combinations thereof.

7. The method of claim 1, wherein the protozoans, bacteria, or fungal cells are selected from the group consisting of P. falciparum and P. berghei.

8. The method of claim 1, wherein the composition is in a solution, and the solution contains an amount that provides about 37 mM to about 75 mM concentration of the synthetic lysine analog, derivative, mimetic, or prodrug in a subject.

9-10. (canceled)

11. The method of claim 8, wherein the solution is administered intravenously, topically, orally, in a time-released fashion, systemically, via a transdermal patch or via an injected or implanted liposomal delivery depot.

12-13. (canceled)

14. The method of claim 11, wherein the solution is administered every 8 hours.

15. The method of claim 11, wherein the solution is administered as an initial bolus followed by continuous infusion for a requisite period of time.

16. The method of claim 15, wherein the continuous infusion is approximately one-tenth of the initial bolus per hour.

17. The method of claim 11, wherein the solution is administered for approximately 5 to 7 days.

18. The method claim 11, wherein the solution is administered until an infection caused by the protozoans, bacteria, or fungal cells has been resolved.

19. The method of claim 8, wherein the solution is applied as part of a vehicle which adapts to human skin.

20-22. (canceled)

23. The method of claim 19, wherein the solution is administered topically every 8 hours.

24. The method of claim 19, wherein the solution is administered topically until an infection caused by the protozoans, bacteria, or fungal cells has been resolved.

25-29. (canceled)

30. The method of claim 11, wherein the composition is administered every 8 hours.

31. The method of claim 11, wherein the composition is administered for approximately 5 to 7 days.

32-33. (canceled)

34. The method of claim 1, wherein the administering of the composition occurs at least once a day.

35-38. (canceled)

39. The method of claim 1, wherein the administering is performed systemically.

40-43. (canceled)

44. A composition to prevent or inhibit proliferation, growth and formation, or survival of protozoans, bacteria, or fungal cells, the composition comprising:
a synthetic lysine analog, derivative, mimetic, or prodrug,
wherein the synthetic lysine analog, derivative, mimetic, or prodrug disrupts at least one of protein-protein, protein-DNA, or protein-RNA interaction by antagonizing at least one of lysine, arginine, or histidine residues on a protein, inhibits proliferation, growth and formation, or survival of the protozoans, bacteria, or fungal cells by occupying binding sites of lysine residues required for proliferation, growth and formation, or survival of the protozoans, bacteria, or fungal cells, inhibits proliferation, growth and formation, or survival of the protozoans, bacteria, or fungal cells by occupying binding sites of lysine residues required for proliferation, growth and formation, or survival of the protozoans, bacteria, or fungal cells or deprives lysine or arginine from a biosynthetic pathway required by the protozoans, bacteria, or fungal cells to thereby inhibit proliferation, growth and formation, or survival of the protozoans, bacteria, or fungal cells.

45-47. (canceled)

48. The composition of claim 44, wherein the synthetic lysine analog, derivative, or mimetic is selected from the group consisting of tranexamic acid, epsilon-aminocaproic acid (EACA), and AZD 6564.

49-50. (canceled)

51. The composition of claim 44, wherein the synthetic lysine analog, derivative, mimetic, or prodrug is in a solution, and the solution contains an amount that provides about 37 mM to about 75 mM concentration of the synthetic lysine analog, derivative, mimetic, or prodrug in a subject.

52. The composition of claim 51, wherein the solution contains an amount that provides about 37 mM concentration of the synthetic lysine analog, derivative, mimetic, or prodrug in a subject.

53. The composition of claim 51, wherein the solution contains an amount that provides about 75 mM concentration of the synthetic lysine analog, derivative, mimetic, or prodrug in a subject.

54. (canceled)

55. The composition of claim 51, wherein the solution contains an amount that provides up to about 100 mg/kg concentration by weight of a subject of the lysine analog, derivative, mimetic, or prodrug in the subject.

56. The composition of claim 51, wherein the solution contains an amount that provides about 10 mg/kg concentration by weight of a subject of the lysine analog, derivative, mimetic, or prodrug in the subject.

57-62. (canceled)

63. The composition of claim 51, wherein the solution is in a gel or cream formation.

64. The composition of claim 51, wherein the solution has a concentration of about 1 to 30% by weight of the synthetic lysine analog, derivative, mimetic, or prodrug.

65. The composition of claim 51, wherein the solution has a concentration of about 20% by weight of the synthetic lysine analog, derivative, mimetic, or prodrug.

66-69. (canceled)

70. The composition of claim 44, wherein the synthetic lysine analog, derivative, mimetic, or prodrug is in a form of at least one of a pill, a tablet, a capsule, or an oral solution or syrup.

71. The composition of claim 44, comprising the synthetic lysine analog, derivative, mimetic, or prodrug in an amount that provides up to about 200 mg/kg concentration by weight of a subject of the lysine analog, derivative, mimetic, or prodrug in the subject.

72. The composition of claim 44, comprising the synthetic lysine analog, derivative, mimetic, or prodrug in an amount that provides about 20 mg/kg concentration by weight of a subject of the lysine analog, derivative, mimetic, or prodrug in the subject.

73-80. (canceled)

81. The composition of claim 44, further comprising ingredients that provide for rapid systemic penetration or extended release.

82. (canceled)

83. The composition of claim 44, further comprising an additional therapeutic agent.

84. The composition of claim 83, wherein the additional therapeutic agent has a mechanism of action complimentary to the synthetic lysine analog, derivative, mimetic, or prodrug.

85. The composition of claim 44, where the synthetic lysine analog, derivative, mimetic, or prodrug is a lysine prodrug that causes production of lysine, a lysine analog, a lysine derivative, or a lysine mimetic in a subject.

86. The composition of claim 44, wherein the synthetic lysine analog, derivative, mimetic, or prodrug is a lysine prodrug selected from the group consisting of a lysine analog prodrug, a lysine derivative prodrug, and a lysine mimetic prodrug.
